(19) United States
(12) Patent Application Publication
(10) Pub. No.: US 2014/0234212 A1
(43) Pub. Date: Aug. 21, 2014

(54) Targeted Buccal Delivery of Agents

(71) Applicant: Massachusetts Institute of Technology, Cambridge, MA (US)

(72) Inventors: Manijeh Nazari Goldberg, Newburyport, MA (US); Maria Jose Alonso, Santiago de Compostela (ES); Kuan-Ju Chen, Cambridge, MA (US)

(73) Assignee: Massachusetts Institute of Technology, Cambridge, MA (US)

(21) Appl. No.: 14/186,977

(22) Filed: Feb. 21, 2014

Related U.S. Application Data
(60) Provisional application No. 61/767,589, filed on Feb. 21, 2013.

(51) Int. Cl. A61K 9/00; A61K 47/36; A61K 33/24

(52) U.S. Cl. CPC A61K 9/006; A61K 33/24; A61K 47/36
USPC 424/1.25; 424/484; 424/9.3; 424/649; 424/488

(57) ABSTRACT

A delivery device for topical and systemic delivery of agents to targeted oral locations, such as mouth cancer cells, has been developed. The formulation includes a mucoadhesive polymeric matrix such as chitosan, which contains one or more therapeutic and/or diagnostic agents, taste masking agents, permeation enhancers, the therapeutic or diagnostic agent to be delivered, and a hydrophilic polymeric coating such as polyethyleneglycol (“PEG”). In the preferred embodiment, the matrix is formulated with one side having the PEG-mucoadhesive polymer exposed for topical placement onto epithelial or cancer cells in the mouth or other mucosal area and the side(s) facing the inside of the oral cavity being covered with a biocompatible, inert membrane that is impermeable to the therapeutic and/or diagnostic agent(s) to be delivered.

### CROSS-REFERENCE TO RELATED APPLICATIONS

This application claims priority to U.S. Ser. No. 61/767,589, filed Feb. 21, 2013.

### FIELD OF THE INVENTION

This invention is generally in the field of formulations for targeted delivery of agents to the oral mucosa, for example, of antitumor agents for treatment of oral cancer.

### STATEMENT REGARDING FEDERALLY SPONSORED RESEARCH

The U.S. government has no rights in this invention.

### BACKGROUND OF THE INVENTION

According to the Oral Cancer (OC) Foundation, about 40,000 Americans will be diagnosed with oral and pharyngeal cancer this year alone, causing 8,000 deaths, killing roughly one person per hour, 24 hours per day. The problem is significantly greater worldwide; with over 640,000 new cases each year. While the incidence of many cancers is decreasing, the incidence of OC has been increasing five years in a row. Currently, cisplatin, cis-diamminedichloroplatinum(ll) (CIS), is the most common antitumor treatment for OC.

The problems with systemic agent delivery are well known. Side effects due to the high dosage requirements and inadvertent treatment of regions of the body not requiring treatment, especially in the case of cancer where the agents are almost as bad as the disease, create a strong need for local administration of agents.

Only half of these OC patients will be alive five years after diagnosis. In certain countries, such as Sri Lanka, India, Pakistan, and Bangladesh, OC is the most common cancer. In parts of India, it represents more than 50% of all cancers. In the US alone, OC incidences increased by 11% between 2002 and 2007. By 2020, the annual worldwide incidence is predicted to increase to over 840,000, a 30% rise, and the annual mortality to increase to nearly 480,000, approximately a 37% increase.

According to the OC Foundation, any oral lesion that lasts more than three weeks needs to be checked by a clinician and treated. There is no safe, effective and convenient treatment accessible to patients. If the toxicity of chemotherapy agents is significantly reduced, clinicians will be more likely to treat the patients with low doses as early as possible.

Local delivery of a therapeutic to the mouth is very difficult. The mucosa forms a formidable barrier to adhesion and few things can penetrate this viscous, slippery material to reach the oral epithelial cells underneath, much less stay attached to the cells long enough to deliver an effective amount of the therapeutic agent.

As reported by Lai, et al., Adv Agent Deliv Rev., 27: 61(2): 158-171 (2009), mucus is a viscoelastic gel layer that protects tissues that would otherwise be exposed to the external environment. Mucus is composed primarily of crosslinked and entangled mucin fibers secreted by goblet cells and submucosal glands. Mucins are large molecules, typically 0, 5-40 MDa in size formed by the linking of numerous mucin monomers, each about 0.3-0.5 MDa, and are coated with a complex and highly diverse array of proteoglycans. At least twenty mucin-type glycoproteins have been assigned to the MUC gene family, with several mucin types expressed at each mucosal surface. Mucins can be generally separated into two families-cell-associated mucins ranging between 100-500 nm in length that contain a transmembrane domain, and secreted mucins that are up to several microns long. Individual mucin fibers are roughly 3-10 nm in diameter, as determined by biochemical and electron microscopy studies. They are highly flexible molecules, with a persistence length of roughly 15 nm. With the exception of specific disease states (such as COPD and CF), the mucin content ranges between 2-5% by weight for cervical, nasal, and lung mucus, with glycosylated oligosaccharides representing 40-80% of the mucin mass. The water content in most mucus types (i.e., lung, gastric, cervicovaginal) commonly falls within the 90-98% range. In addition to mucins, mucus gels are loaded with cells, bacteria, lipids, salts, proteins, macromolecules, and cellular debris. Mucus pH can vary greatly depending on the mucosal surface, with highly acidic environments capable of aggregating mucin fibers and greatly increasing the mucus viscoelasticity. Lung and nasal mucus are in general pH neutral and eye mucus is slightly basic with pH ˜7.8. In contrast, gastric mucus is exposed to a wide range of pH: a large pH gradient exists within the same mucus cross-section, with pH rising from the luminal pH of ˜1-2 to ˜7 at the epithelial surface. Vaginal secretions typically exhibit pH in the range of 3.5 to 4.5 due to acidification from lactic acid produced by lactobacilli under anaerobic conditions. Beyond biochemical differences, the thickness of the mucus blanket also varies for different mucosal surfaces. The nasal tract, which has a mucus layer of limited thickness, is readily accessible and considered highly permeable compared to other mucosal surfaces. In the human GI tract, the mucus layer is thickest in the stomach and the colon, but exhibits significant variation.

Mucus is continuously secreted, then shed and discarded or digested and recycled. Its lifetime is short, often measured in minutes to hours. The understanding of mucus layer thickness and clearance times at various mucosal surfaces is important to the development of particles designed to overcome mucosal clearance mechanisms, since they must penetrate mucus at rates markedly faster than mucus renewal and clearance in order to overcome the barrier. Little is known about the oral mucosa.

Delivery of agents is classified into three categories within the oral mucosal cavity: (i) sublingual delivery, for systemic agent delivery through the mucosal membranes lining the floor of the mouth, (ii) buccal delivery, which is through the lining of the cheeks (buccal mucosa) for agent delivery, and (iii) local oral delivery, which is agent delivery into the oral cavity.

Delivery to the oral tissue is difficult since the therapeutic agent that is not applied directly to the epithelial cells is lost through swallowing. Only the area of contact can form an effective conduit for the agent reaching the oral epithelial cells. Taste is also a major challenge in agent delivery to this region. Taste masking of agents is an important factor in the design of delivery means. Another major difficulty with delivering therapeutics through the oral mucosa verses other mucosa such as inside the intestine is that the epithelium of the oral cavity is about 40 to 50 cell layers deep, with tight junctions that prevent permeation of agents. By contrast, the epithelium of the intestine is only a single cell layer.

It is therefore an object of the present invention to provide a therapeutic or diagnostic delivery device for local and systemic administration through the oral epithelial cells that can penetrate the thick tight oral epithelium.

It is also an object of the present invention to provide a therapeutic or diagnostic delivery device that taste masks the therapeutic or diagnostic agent to be delivered.

It is another object of the present invention to provide a therapeutic or diagnostic delivery device that is effective despite the constant saliva and washout problems associated with oral delivery.

It is a further object of the present invention to provide a method for administering a therapeutic or diagnostic agent to a specific region of the oral epithelia.

### SUMMARY OF THE INVENTION

A delivery device for topical and systemic delivery of agents to targeted oral locations, such as mouth cancer cells, has been developed. The formulation includes a mucoadhesive polymeric matrix, which contains one or more therapeutic and/or diagnostic agents, taste masking agents, permeation enhancers and agent-encapsulated nanoparticles. The nanoparticles are formed of a mucoadhesive polymer such as a chitosan or cyclodextrin, optionally in combination with targeting molecules such as RGD peptides, folate, antibody or glucose analogs; the therapeutic or diagnostic agent to be delivered, and a hydrophilic polymeric coating such as polyethyleneglycol (“PEG”) to enhance penetration through the mucosa to the site for delivery. In one embodiment, the formulation includes chitosan or cyclodextrin nanoparticles for delivery of cisplatin (“CIS”) or other chemotherapeutic agent or anti-inflammatory agents, a polyethylene glycol (PEG) coating to enhance mucosal penetration of the nanoparticles, and targeting motif (RGD peptides or glucose analog) attached to the PEG to increase bioadhesion to targeted cells. The mucoadhesive polymer is used to taste mask the therapeutic agent while retaining it at the site of the cancer cells. Large loading dosages can be achieved, for example, a loading of 20% cisplatin.

In the preferred embodiment, the matrix is formulated with one side having the PEG-mucoadhesive polymer exposed for topical placement onto epithelial or cancer cells in the mouth or other mucosal area and the side(s) facing the inside of the oral cavity being covered with a biocompatible, inert membrane that is impermeable to the therapeutic and/or diagnostic agent(s) to be delivered. The matrix can include additional components, such as taste-masking agents to prevent the bitterness and unpleasant taste of the therapeutic agents, for example, citric acid or other fruity flavoring; permeation enhancers, for example, PEG, bile salt, citric acid or others; and anti-inflammatory or anti-oxidant agents, for example, curcumin.

### BRIEF DESCRIPTION OF THE DRAWINGS

FIG. 1 is a diagram of the delivery device, manufacture and use.

FIG. 2 is a graph showing encapsulation efficiency at different percent loading capacities for cisplatin-loaded nanoparticles.

FIG. 3 is a graph showing the size and charge of the cisplatin-loaded nanoparticles at different pH.

FIG. 4 is a graph showing in vitro release profile of cisplatin-loaded nanoparticles at pH 6.

FIG. 5 is a graph showing release over time, of cisplatin-encapsulated nanoparticles from a chitosan sponge.

FIG. 6A shows the viability of FaDu cells in response to different concentrations of cisplatin-loaded nanoparticles (), blank-nanoparticles (▪), free cisplatin (▴) or without treatment for 24 h (▾). FIG. 6B shows the viability of FaDu cells in response to different concentrations of cisplatin-loaded nanoparticles (), blank-nanoparticles (▪), free cisplatin (▴) or without treatment for 48 h (▾). FIG. 6C shows the viability of HCPC1 cells in response to different concentrations of cisplatin-loaded nanoparticles (), blank-nanoparticles (▪) or free cisplatin (▴) for 48 h.

FIGS. 7A and 7B show the viability of KB cells exposed to cisplatin-loaded nanoparticles (15%) for 24 h (FIG. 7A) or 72 h (FIG. 7B).

FIG. 8 shows the in vivo therapeutic efficacy study of cisplatin-loaded nanoparticles using FaDu cell xenografting mouse model.

FIGS. 9A and 9B are graphs of a tumor inhibition study of hamster cheek pouch carcinoma (HCPC1) cell line allografted hamsters treated with CIS-NPs embedded sponge topically and free cisplatin intraperitoneally. The results show completed tumor elimination of CIS-NPs embedded sponge treated hamster after four treatments (FIG. 9A). The weight changing plot of the HCPC1 allografted hamsters over the course of the treatments shows insignificant weight loss compared to the healthy group; whereas the free cisplatin intraperitoneal group shows significant weight loss (FIG. 9B).

FIG. 10 is a graph of cellular uptake of nps by TR-146 cells.

FIG. 11 is Thiol-modified chitosan polymer.

FIG. 12 is Fluorophore conjugated chitosan.

### DETAILED DESCRIPTION OF THE INVENTION

A delivery device has been developed specifically for delivery within the oral cavity to the oral mucosa. Requirements for the delivery device include:

The device has to adhere to the buccal tissue regardless of the biofilm

The device has to have a powerful taste masking element for patient compliance

The device has to prevent the agent from being washed down the throat

For systemic delivery, the device must have sufficient permeation ability to permeate through approximately 50 layers of cells prior to reaching the systemic circulation.

For local delivery, the permeation has to be adjustable to the desired depth.

### I. DEFINITIONS

“Kilo count per second” (Kcps)”, mean count rate (in kilo counts per second (kcps)). If the count rate of the sample is lower than 100, the measurement should be aborted meaning the concentration of the sample is too low for measurements. A sample with suitable Kcps can be considered a stable sample with idea concentration for measurement.

“Polydispersity index” (PDI) or simply, “dispersity” is used herein to refer to a measure of the heterogeneity of sizes of particles in a mixture. PDI measures the size dispersity of nanoparticles.

“Zeta potential” (ZP) is used herein to refer to the overall charge that nanoparticles acquires in a particular medium and can be measured on a Zetasizer Nano instrument.

“Mucoadhesive” is a property of a material that has the ability to adhere to mucosal membranes in the human body.

“Biocompatible” refers to the ability of a biomaterial to perform its desired function with respect to a medical therapy, without eliciting any significant undesirable local or systemic effects in the recipient or beneficiary of that therapy, but generating the most appropriate beneficial cellular or tissue response in that specific situation, and optimizing the clinically relevant performance of that therapy.

“Biodegradable” refers to a property of the materials that is capable of being broken down especially into innocuous products by the action of living things.

### I. Agent Delivery Devices

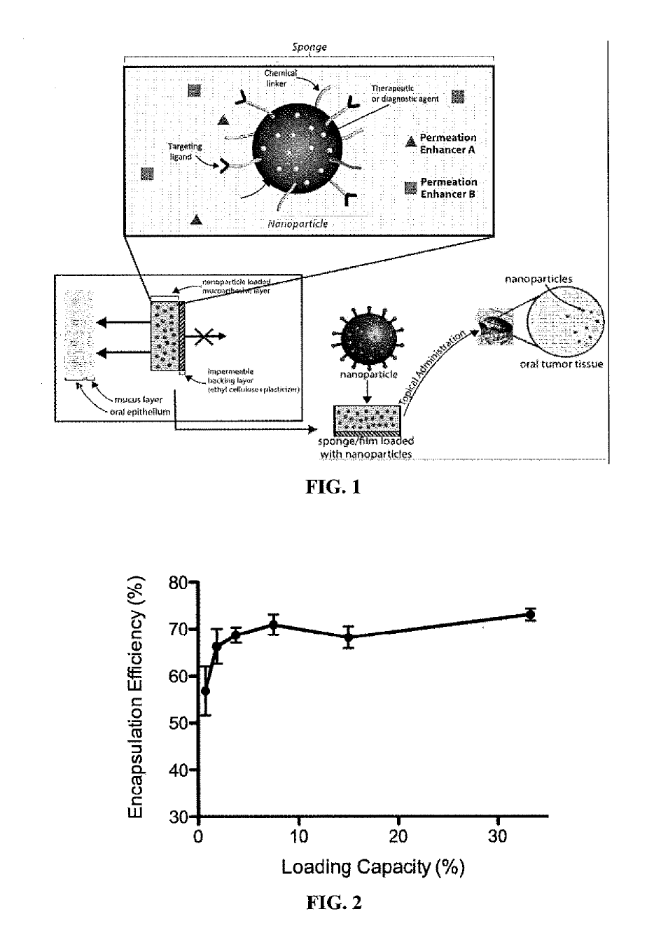

A representative delivery device is shown in FIG. 1.

The device 10 includes a nanoparticle loaded mucoadhesive matrix 12 and an impermeable backing layer 14. The nanoparticles 16 are dispersed in the mucoadhesive matrix 12. The nanoparticles 16 include a polymer 18, having dispersed or encapsulated therein a therapeutic, prophylactic, diagnostic or nutraceutical agent 20. Optionally, the nanoparticles 16 can include chemical linkers 22, which may couple targeting ligands 24 and/or additional agent 20 to the nanoparticles 16. The mucoadhesive matrix 12 can include one or more penetration enhancers 26a, 26b.

The mucoadhesive matrix 12 of the device 10 is applied to the oral cavity, preferably onto a mucus layer 30, allowing delivery of the agent 20 to the underlying oral epithelium 32. The nanoparticles 16 penetrate the mucosa and release agent 20 directly into the tissue. Targeting ligands 24 are used for preferential delivery, such as to tumor cells 34.

The matrix is formed of bioadhesive polymer, most preferably a mucoadhesive polymer. The matrix has nanoparticles dispersed therein, and may include a plasticizer. A bioadhesive polymer well known for mucoadhesiveness and ability to combine a polymeric delivery system and taste masking agents into a porous architecture is selected for the matrix. A membrane that is inert and impermeable to agent diffusion is applied to the surface which is not used for agent delivery. This yields a mucoadhesive material with a uni-directional delivery of agent. Permeation enhancers and taste masking agents can be added to enhance penetration of the agent.

The nano-scale size particles permeate the tissue of the oral cavity. NPs with a size below 200 nm penetrate through mucosa and are taken up by cancer cells. This size is adequate to carry enough therapeutic or diagnostic agent such as a chemotherapeutic like cisplatin (CIS) to obtain high loading and encapsulation efficiencies (higher than 80%), which is desirable for scaling up and commercialization.

The encapsulation protects the taste buds in the mouth from the unpleasant metallic flavor of agents such as cisplatin, allows for a coating of a hydrophilic polymer such as PEG for controlled penetration into the mucus, allows for adding a targeting ligand, and allows for controlled release of agent. Moreover, in the case of systemic penetration, the encapsulation also reduces uptake by the body's reticuloendothelial system. The smaller particles have greater surface area-to-volume ratios, which cause the particles' dissolution rates to be higher than that of larger particles, enabling them to overcome permeation limits due to solubility factors. The large surface area to volume increases the bioadhesivity. These factors in combination result in penetration of the agent deep into the cancer cells, providing a definite benefit.

There are three main aspects of targeting that help achieve localized delivery:

1. Direct: Achieved by placing the agent-loaded nanoparticles directly on the cancer tumor.
2. Active: Obtained using molecular targeting agents, such as RGD, to further focus on cancer cells and reduce toxicity on healthy cells.
3. Passive: Occurs naturally due to the vascular architecture in tumors that causes higher agent uptake in the tissue. This is known as the enhanced permeability and retention effect (EPR).

A. Mucoadhesive Polymeric Nanoparticles

A number of bioadhesive and mucoadhesive polymers are known. In the preferred embodiment, the polymer is mucoadhesive so that it can bind to a mucosal region of the oral cavity. Preferably, the polymer is polycationic, biocompatible, and biodegradable. The preferred polymer is chitosan.

Chitosan is a polycationic, non-toxic, biocompatible and biodegradable polymer. In addition, chitosan has different functional groups that can be modified with a wide array of ligands. Because of its unique physicochemical properties, chitosan has great potential in a range of biomedical applications. Chitosan (CHI) has been commonly used as a mucosal agent delivery mechanism because of its bio-adhesiveness and permeability properties. The barrier in oral epithelium can easily be disrupted by chitosan particles, enhancing permeability through buccal mucosa.

The primary amine groups of chitosan can be utilized for chitosan modification through biotinylation using N-hydroxysuccinimide chemistry. This is followed by the addition of avidin which strongly binds to biotin. Biotinylated ligands such as polyethylene glycol (PEG) and RGD peptide sequence, or biotinylated enzymes can then be added to modify the surface properties of the chitosan. Different factors affect fabrication of chitosan particles, such as the pH of the preparation, the inclusion of polyanions, the charge ratios and the degree of deacetylation and the molecular weight of chitosan.

Chitosan nanoparticles are preferred for use with chemotherapeutics such as doxorubicin because of the chitosan's sensitivity to low pH since cancer tissue is acidic, and the particles release the agent faster in an acidic environment. The controlled release of the agent from the chitosan nanoparticles insures that a steady amount of agent targets the cancer tissues while minimizing toxic side effects to the surrounding healthy tissues.

Other useful natural polymers are cyclodextrin and pectin. Several studies report on synthetic bioadhesive polymers. Polyamides, polycarbonates, polyalkylenes, polyalkylene glycols, polyalkylene oxides, polyalkylene terephthalates, polyvinyl ethers, polyvinyl esters, polyvinyl halides, polyphosphazines, polyacrylamides, poly(vinyl alcohols), polysiloxanes, polyvinylpyrrolidone, polyglycolides, polyurethanes, polystyrene, polyvinylphenol, polymers of acrylic and methacrylic esters, polylactides, copolymers of polylactides and polyglycolides, poly(butic acid), poly(valeric acid), poly(lactide-co-caprolactone), poly[lactide-co-glycolide], polyanhydrides, polyorthoesters, blends and copolymers thereof are described by U.S. Pat. No. 6,217,908 by Mathiowitz, et al. Nafee, et al., Drug Dev. Ind Pharm., 30(9):985-983 (2004), reported that carbopols (CP934, and CP940), polycarbophil (PC), sodium carboxymethyl cellulose (SCMC) and, anionic polymers, chitosan (Ch) as a cationic polymer and hydroxypropylmethyl cellulose (HPMC) as a non-ionic polymer, were all useful, but that polyacrylic acid derivatives (PAA) showed the highest bioadhesion force, prolonged residence time and high surface acidity. SCMC and chitosan also had good bioadhesive characteristics, while HPMC and pectin exhibit weaker bioadhesion.

B. Hydrophilic Polymer Enhancing Mucosal Penetration

In the preferred embodiment, a dense coating of low-molecular weight polyethylene glycol (PEG), most preferably about 5000 Daltons, or PLURONIC®, nonionic triblock copolymers composed of a central hydrophobic chain of polyoxypropylene (poly(propylene oxide)) flanked by two hydrophilic chains of polyoxyethylene (poly(ethylene oxide)), sold by BASF, is covalently attached onto a chitosan nanoparticle surface to make the particles more hydrophilic and thereby penetrate mucosa more easily. Other useful polymeric enhancers of mucosal epithelial permeability were reported by Di Colo, et al., J. Pharm. Sci., 97(5):1652-1680 (2008). Active polymers can be classified into: polycations (chitosan and its quaternary ammonium derivatives, poly-L-arginine (poly-L-Arg), aminated gelatin), polyanions (N-carboxymethyl chitosan, poly(acrylic acid)), and thiolated polymers (carboxymethyl cellulose-cysteine, polycarbophil (PCP)-cysteine, chitosan-thiobutylamidine, chitosan-thioglycolic acid, chitosan-glutathione conjugates).

C. Tissue Adherent Molecules

Numerous tissue targeting moieties are known. Targeting moieties are classified as proteins (mainly antibodies and their fragments), peptides, nucleic acids (aptamers), small molecules, or others (vitamins or carbohydrates). Although monoclonal antibodies (mAbs) have been widely used as escort molecules for the targeted delivery of nanoparticles, several limitations including large size and difficulty in conjugation to nanoparticles have hampered their use. Thus, other smaller-sized ligands including peptides are used when possible. Peptide-based targeting ligands may be identified via several methods. Most commonly, they are obtained from the binding regions of a protein of interest. Phage display techniques can also be used to identify peptide-targeting ligands. In a phage display screen, bacteriophages present a variety of targeting peptide sequences in a phage display library (˜10¹¹ different sequences), and target peptides are selected using a binding assay. Cilengitide, a cyclic peptide with integrin binding affinity, is currently in phase II clinical trials for the treatment of non-small cell lung cancer and pancreatic cancer. Adnectin for human VEGF receptor 2 (Angiocept), a 40 amino acid thermostable and protease-stable oligopeptide, entered phase I clinical trials for the treatment of advanced solid tumors and non-Hodgkin's lymphoma in 2006. Although peptides can have drawbacks, such as a low target affinity and susceptibility to proteolytic cleavage, these issues may be ameliorated by displaying the peptides multivalently or by synthesizing them using D-amino acids. See Yu, et al., Theranostics. 2(1): 3-44 (2012). See also Snook et al., Cancer Immunology, Immunotherapy 61(5):713-723 (2012) for mucosal epithelial specific epitopes that can be targeted.

One of the cancer cell targeting moieties is RGD, a tumor vasculature homing peptide that targets integrin receptors. It is a cell adhesion protein that is highly expressed during angiogenesis (blood vessel formation) and is critical to tumor proliferation. RGD bound directly to chitosan nanoparticles to increase tissue adhesion for agent delivery is known (Han, et al., Clin Cancer Res., 16:3910 (2010)) and bound to chitosan-PEG particles to increase delivery of chemotherapeutics to tumors is known (Lv, et al., Mol. Pharmaceutics, 9:1736-1747 (2012)).

Integrins, α(2)β(1) and α(3)β(1), can be recognized by RGD. The data also suggest RGD sequence can recognize at least 12 of the integrin heterodimers and, indeed, in oral SCC cell lines, α(2)β(1) and α(3)β(1) are highly expressed and this results in great transfection efficiency.

The surface coverage of RGD on the nanoparticles can be adjusted by varying the ratio of PEG chain and PEG-RGD chain on the surface of nano particles. Targeting specificity and delivery performance of the nano particles can be affected by the density of RGD motif. In general, a RGD coverage of 5-10% is considered as effective.

Biomarkers which can be used for targeting oral cancer are described in the literature, for example, Hsu, et al. Mol Cancer Res., 10(11):1430-9 (2012). The studies show that IL-20 promoted oral tumor growth, migration, and tumor-associated inflammation, which can be a target for treating oral cancer. IL-6 is another specific marker for oral squamous carcinoma (Culig, Expert Opin Ther Targets, 17(1):53-9 (2013).

Anti-IL-20 or anti-IL-6 monoclonal antibody can be conjugated onto the end of PEG chain as an alternative targeting motif to increase the specificity and efficacy of the delivery system.

¹⁸F-FDG (2-deoxy-2-[¹⁸F]fluoro-d-glucose) PET (positron emission tomography) is a functional imaging technique that provides information about tissue metabolism and has been successfully applied to the evaluation of head and neck cancer (Nakagawa, et al., J Nucl Med., 49(7):1053-1059 (2008)). The glucose analog ¹⁸F-FDG is transported into cells by facilitative glucose transporters (Mueckler, Eur J Biochem., 219:713-725 (1994)). Overexpression of ubiquitous glucose transporter type 1 (GLUT1) in malignant tumors allows ¹⁸F-FDG PET to have a useful role in oncology (Smith, Br J Biomed Sci., 56:285-292 (1999).

Glucose analogs are useful as a targeting motif to cancer cells as a result of their role in cancer cells metabolism. Various glucose analogs have been developed, such as 1-thio-β-D-glucose, which has been demonstrated to be a specific probe for melanoma (Castelli, et al., Current Radiopharmaceuticals, 4(4):355-360 (2012); and D-Glucosamine(2-amino-2-deoxy-D-glucose) hydrochloride, which has been conjugated onto iron oxide nanoparticles for Hela cells targeting in vitro (Xiong, et al., Pharm Res., 29:1087-1097 (2012)).

By conjugating glucose analogs with different functional group substituted for the normal hydroxyl group at either 1 or 2′ position in the glucose molecule-(1 or 2)-(functional group)-β-D-glucose); for example: 1-Azido-β-D-glucose, 1-Azido-β-D-glucose tetraacetate, 2-Azido-β-D-glucose, 2-Azido-β-D-glucose tetraacetate, 1-thio-β-D-glucose, 2-thio-β-D-glucose) onto the PEG chain of PEGylated Chitosan Nanoparticles, the targeting specificity of oral cancer can be improved.

D. Biocompatible Impermeable Membrane or Coating

The matrix is coated by spraying, dipping or contact with a membrane or impermeable coating such as those used to back bioadhesive tablets and device. See, for example, Boddupalli, et al., J Adv Pharm Technol Res., 1(4): 381-387 (2010); Robinson and Irons; Handbook of Adhesive Technology, Ed A. Pizzi and K. L. Mittal (CRC Press 2003).

The non-permeable backing performs a dual function: (1) prevents agent loss and (2) provides agent taste masking.

In general, impermeable coatings are formed of polymers such as cellulose acetate, hydroxypropylmethylcellulose or Eudragit (polyacrylamides). These may also be bioadhesive. Biodegradable materials are preferred due to the likelihood the material could be swallowed.

E. Agents to be Encapsulated

Any therapeutic, prophylactic, diagnostic or nutraceutical agent may be encapsulated. Representative agents include chemotherapeutics, antiinfectives, antibiotics, antifungals, antivirals, anti-inflammatories, immunomodulators, vaccines, and combinations thereof.

A preferred agent is a platinum based chemotherapeutic such as cisplatin (CIS), Historically, CIS has been at the forefront of platinum based chemotherapeutics and is a standard of care in the treatment of many cancers including OC. While CIS is a leading therapy for many cancers, it is often hindered by its significant systemic toxicity as a result of traditional bolus systemic IV doses. This system avoids that toxicity.

Diagnostic agents may be radiopaque, radioactive or other. For example, a diagnostic agent may be an imaging agent such as iron oxide, gadolinium complex, radioisotopes, gold and combinations thereof.

F. Additional Additives

Other compounds that can be included are tastemasking agents such as flavorings (mint, bubblegum, orange or citrus flavorings, etc.) and antioxidants that are useful to prevent bacterial contamination. A representative anti-angiogenic agent is curcumin and purified components thereof, antibiotics such as tetracycline and derivatives thereof, as well as chemotherapeutics such as thalidomide. These may also help with treating cancer or infection at the site of treatment.

These agents may be coated onto, disperse within, or encapsulated within the matrix or nanoparticles.

### II. METHODS OF MANUFACTURE

There are at least four methods available to make chitosan particles: ionotropic gelation, microemulsion, emulsification solvent diffusion and polyelectrolyte complex. The most widely developed methods are ionotropic gelation and self-assembling polyelectrolytes. These methods offer many advantages such as simple and mild preparation method without the use of organic solvent or high shear force. They are applicable to a broad categories of agents including macromolecules which notorious as labile agents. In general, the factors found to affect nanoparticles formation including particle size and surface charge are molecular weight and degree of deacetylation of chitosan. The entrapment efficiency is found to be dependent on the pKa and solubility of entrapped agents.

The ionotropic gelation method is commonly used to prepare chitosan nanoparticles. In an acidic solution, the amine group of chitosan molecule is protonized and interacts with an anion such as tripolyphosphate (TPP) by ionic interaction to form particles (Lee, et al., Polymer, 42:1879-1892 (2001)). This method is very simple and mild. Reversible physical crosslinking by electrostatic interaction, instead of chemical crosslinking, is applied to prevent possible toxicity of reagents and other undesirable effects (Shu, et al., Internat. J. Pharm., 201:51-58 (2000)).

See also Lv, et al., Mol. Pharmaceutics, 9:1736-1747 (2012), describing a tumor targeting delivery system for insoluble agent (paclitaxel, PTX) by PEGylated O-carboxymethyl-chitosan (CMC) nanoparticles grafted with cyclic Arg-Gly-Asp (RGD) peptide. To improve the loading efficiency (LE), a O/W/O double emulsion method was combined with temperature-programmed solidification technique and controlled PTX within the matrix network as in situ nanocrystallite form. Furthermore, these CMC nanoparticles were PEGylated, which could reduce recognition by the reticuloendothelial system (RES) and prolong the circulation time in blood.

Methods of making chitosan particles by microemulsion are known. For example, an amphiphilic graft copolymer using chitosan (CS) as a hydrophilic main chain and poly(lactic-co-glycolic acid) (PLGA) as a hydrophobic side chain is prepared through an emulsion self-assembly synthesis. CS aqueous solution is used as a water phase and PLGA in chloroform serves as an oil phase. A water-in-oil (W/O) emulsion is fabricated in the presence of the surfactant span-80. The CS-g-PLGA amphiphile can self-assemble to form micelles with size in the range of ≈100-300 nm, which makes it easy to apply in various targeted-drug-release and biomaterial fields. Chitosan can be dissolved into deionized water together with 1-hydroxybenzotriazole. A water-in-oil (W/O) chitosan and poly(lactic-co-glycolic acid) microemulsion is prepared and then a chitosan-graft-poly(lactic-co-glycolic acid) stimuli-responsive amphiphile is fabricated. The obtained amphiphile can self-assemble to form micelle in suitable solvents. Cai, Int J Nanomedicine, 6:3499-508 (2011), describes RGD peptide-mediated chitosan-based polymeric micelles targeting delivery for integrin-overexpressing tumor cells.

Chitosan microparticles can be prepared by the water-in-oil emulsion solvent diffusion method. Chitosan solution is added drop-wise to ethyl acetate with stirring for 45 min. After emulsification-diffusion, the chitosan microparticles are recovered by centrifugation and dried in a vacuum oven at 30° C. for 24 h.

The cationic amino groups on the C2 position of the repeating glucopyranose units of chitosan can interact electrostatically with the anionic groups (usually carboxylic acid groups) of other polyions to form polyelectrolyte complexes. Many different polyanions from natural origin (e.g. pectin, alginate, carrageenan, xanthan gum, carboxymethyl cellulose, chondroitin sulphate, dextran sulphate, hyaluronic acid) or synthetic origin (e.g., poly (acrylic acid)), polyphosphoric acid, poly (L-lactide) have been used to form polyelectrolyte complexes with chitosan in order to provide the required physicochemical properties for the design of specific drug delivery systems (Berger et al. Eur J Pharm Biopharm. 2004; 57:35-52).

As demonstrated by Example 5, a chitosan sponge can be prepared by making a solution of chitosan, adding acid, then freezing and lyophilizing the chitosan. In a preferred embodiment, nanoparticles containing drug are suspended in the chitosan solution, to yield a chitosan sponge having drug nanoparticles dispersed therein.

### II. METHODS OF ADMINISTRATION

Two commonly reported dosing regimens for oral cancer, assuming an average physiological profile, yield therapeutic concentrations in the range of 15-40 μg/ml. These concentration ranges can be obtained using a NP delivery system. However, it is important to understand the bioavailability of the agent as it is absorbed through the oral mucosa. The intent is to deliver the agent to local tissue, which has direct implications in the total concentration of agent to be delivered. A concentration less than what is given with systemic therapeutic agent is required. For example, CIS is traditionally delivered as an intravenous bolus dose, and is limited by its systemic toxicity. This toxicity notably causes nephrotoxicity (kidney) and ototoxicity (auditory), as well as myelosuppression, nausea, and vomiting. Neurotoxicity is observed with cumulative doses of 200 mg/m². A recent liposomal form of CIS was found to have a maximum tolerated dose of 300 mg/m². Assuming an average physiological profile, plasma concentrations should be less than 76 μg/ml. By keeping cumulative doses in the reported tolerated range this approach avoids systemic toxicity and its associated effects. This should also avoid systemic toxicity if the agent reaches the gastrointestinal (GI) tract as a result of salivary washout. Most agent that ends up in the GI tract will be excreted, as the absorption in the GI tract of most platinum-based anticancer agents is low.

The present invention will be further understood by reference to the following non-limiting examples.

### Example 1

### Preparation of Drug-Free Chitosan-Based Nanoparticles

Nanoparticles were prepared using low molecular weight research grade chitosan as described below.

Materials and Methods

Briefly, 5 mL of tripolyphosphates (TPP) solution in purified water at various w/v (0.1%-1%) was added to a 10 mL acetic acid solution (0.175% v/v) containing various concentrations of low molecular weight, medium weight, and deacetylated chitosan (0.1%-1% w/v), while stirring vigorously. The mixture was continuously stirred under room temperature for 10 min, yielding a collection of chitosan-nanoparticles (CHI-NP) with various sizes.

Nanoparticles were characterized. Size, polydispersity index (PDI), Kilocount per second (KCPS) and zeta potential (ZP) of nanoparticles were measured by Zetasizer Nano (Malvern Instruments, Ltd., UK).

Results

Table 1 summarizes the properties of the nanoparticles prepared from low molecular weight chitosan. By far the most important physical property of particulate samples is particle size. Measurement of particle size distributions is routinely carried out across a wide range of industries and is often a critical parameter in the manufacture of many products. NPs must be below 200 nm to penetrate through mucosa and to be taken up by cancer cells.

As described in Heurtault, et al., Biomaterials, 24:4283-4300 (2003), Zeta potential is an important and useful indicator of particle surface charge, which can be used to predict and control the stability of colloidal suspensions or emulsions Almost all particles in contact with a liquid acquire an electric charge on their surface. The electric potential at the shear plane is called the zeta potential. The shear plane is an imaginary surface separating the thin layer of liquid (liquid layer constituted of counter-ions) bound to the solid surface in motion. The greater the zeta potential the more likely the suspension is to be stable because the charged particles repel one another and thus overcome the natural tendency to aggregate. The measurement of the zeta potential allows predictions to be made about the storage stability of a colloidal dispersion.

The charge of the nanoparticle is critical for their mucoadhesiveness property (Biol Pharm Bull. 2003 May; 26(5):743-6. The correlation between zeta potential and mucoadhesion strength on pig vesical mucosa.). The positive charged nanomaterials have better mucoadhesion strength. Therefore, the required zeta potential is positive charge within a range of 10-50 mV, which is regarded as stable.

TABLE 1
Low Molecular Weight Chitosan Nanoparticles.

| | | Diameter | | |
|---|---|---|---|---|
| Sample Name | KCPS | (nm) | PDI | ZP (mV) |
| 0.1% TPP + 0.1% | 423.6 | 193.2 | 0.283 | 48.24 |
| Chitosan | | | | |
| 0.1% TPP + 0.5% | 213.9 | 810.6 | 0.331 | 67.89 |
| Chitosan | | | | |
| 0.1% TPP + 1% | 308.6 | 2616.3 | 0.383 | −1.42E−01 |
| Chitosan | | | | |
| 0.5% TPP 0.1% | 378.2 | 5721 | 0.289 | 7.63 |
| Chitosan | | | | |
| 0.5% TPP + 0.5% | 456.8 | 990.3 | 0.429 | 65.63 |
| Chitosan | | | | |
| 0.5% TPP + 1% | 54.7 | 4744 | 0.444 | 76.78 |
| Chitosan | | | | |
| 1% TPP + 0.1% | 319.4 | 6391.4 | 0.628 | 2.54 |
| Chitosan | | | | |
| 1% TPP + 0.5% | 465.3 | 802.5 | 0.005 | 6.36E−01 |
| Chitosan | | | | |
| 1% TPP + 1% Chitosan | 476.1 | 608.7 | 0.139 | −2.30E+00 |

KCPS = kilocount per second
PDI = polydispersity index aka dispersity
ZP = zeta potential

Medium molecular weight research grade chitosan was also used to prepare nanoparticles as described above from low molecular weight research grade chitosan. Nanoparticles were characterized as shown in Table 2.

TABLE 2
Medium Molecular Weight Chitosan Nanoparticles.

| | | Diameter | | |
|---|---|---|---|---|
| Sample Name | KCPS | (nm) | PDI | ZP (mV) |
| 0.1% TPP + 0.1% | 361.9 | 1940.3 | 0.241 | −5.07E−01 |
| Chitosan | | | | |
| 0.1% TPP + 0.5% | 296.7 | 822.7 | 0.319 | 146.42 |
| Chitosan | | | | |
| 0.1% TPP + 1% | 483.4 | 939.4 | 0.217 | −3.99E−01 |
| Chitosan | | | | |
| 0.5% TPP + 0.1% | 534 | 2147.6 | 0.373 | −3.29 |
| Chitosan | | | | |
| 0.5% TPP + 0.5% | 362.7 | 1450.3 | 0.005 | 1.94E+01 |
| Chitosan | | | | |
| 0.5% TPP + 1% | 472.4 | 1098.4 | 0.005 | 124.74 |
| Chitosan | | | | |
| 1% TPP + 0.1% | 347.7 | 3152.4 | 0.339 | 2.02E−01 |
| Chitosan | | | | |
| 1% TPP + 0.5% | 394.6 | 901.9 | 0.005 | 2.95E−01 |
| Chitosan | | | | |
| 1% TPP + 1% Chitosan | 305.5 | 1981.6 | 0.005 | −2.07E+01 |

In a separate experiment, nanoparticles were also prepared as described above, using pharmaceutical grade chitosan: PROTASAN™ UP CL 113 (chitosan chloride). PROTASAN™ UP CL 113 (chitosan chloride) is based on a chitosan where between 75-90 percent of the acetyl groups are removed. The cationic polymer is a highly purified and well-characterized water-soluble chloride salt. The functional properties are described by the molecular weight and the degree of deacetylation. Typically, the molecular weight for PROTASAN™ UP CL 113 (chitosan chloride) is in the 50,000-150,000 g/mol range (measured as a chitosan acetate). The ultra low levels of endotoxins and proteins allow for a big variety of in vitro and in vivo applications.

TABLE 3
PROTASAN ™ UP CL 113 (chitosan chloride) Nanoparticles.

| | | Diameter | | ZP |
|---|---|---|---|---|
| Sample Name | KCPS | (nm) | PDI | (mV) |
| 0.1% TPP + 0.1% Chitosan | 332.6 | 403.2 | 0.447 | 30.24 |
| (0.6:1) | | | | |
| 0.1% TPP + 0.1% Chitosan | 313.9 | 133.5 | 0.221 | 44.38 |
| (0.5:1) | | | | |
| 0.1% TPP + 0.1% Chitosan | 308.6 | 102.3 | 0.243 | 66.89 |
| (0.4:1) | | | | |

By using high purity chitosan (The cationic polymer is a highly purified and well-characterized water-soluble chloride salt.), chitosan nanoparticles of better quality were obtained, as shown in Table 3. The optimal formulation was obtained by optimizing different ratios between the TPP and chitosan solution (0.6:1; 0.5:1; 0.4:1). As shown in Table 3, the best formation is with the ratio of TPP to chitosan ca. 0.5:1, which has the ideal size and zeta potential.

### Example 2

### Preparation and Characterization of Cisplatin-Encapsulated Nanoparticles

Materials and Methods

Cisplatin-loaded nanoparticles were prepared using low molecular weight research chitosan, using different concentrations of cisplatin as described below.

Briefly, 5 mL of tripolyphosphates (TPP) solution at 0.1% w/v containing cisplatin (0.1-2 mg) was added to a 10 mL acetic acid solution (0.175% v/v) containing 0.1% w/v chitosan, while stirring vigorously. The mixture was stirred continuously at room temperature for 10 min, yielding a collection of cisplatin-encapsulated CHI-NP with different cisplatin loading amounts.

The optimal formulation for blank nanoparticles is 5 mL of tripolyphosphates (TPP) solution in purified water at 0.1% w/v was added to a 10 mL acetic acid solution (0.175% v/v) containing 0.1% w/v chitosan, while stirring vigorously. The mixture was continuously stirred at room temperature for 10 min, yielding a chitosan-nanoparticle (CHI-NP) solution containing 113 nm NPs.

Cisplatin-loaded Nanoparticles were prepared as followed. 5 mL of 0.1% w/v tripolyphosphates (TPP) solution containing different amounts of cisplatin (0.1-5 mg) was added to a 10 mL acetic acid solution (0.175% v/v) containing 0.1% w/v chitosan, while stirring vigorously. The mixture was stirred continuously at room temperature for 10 min, yielding a collection of cisplatin-encapsulated CHI-NPs with different loadings of cisplatin.

The amount of cisplatin in cisplatin-loaded nanoparticles was quantified by ICP-AES (Inductively coupled plasma atomic emission spectroscopy), and the loading efficiency calculated.

For stability testing, cisplatin-encapsulated nanoparticles were synthesized and characterized by Zetasizer Nano for size distribution. 5 mL of NP solution was then added into 10 mL of PBS, culture medium and water. The resulting individual solution was then characterized again. The storage stability test of NP solution was carried out after 3 weeks in room temperature.

Results

Nanoparticles prepared as described above were characterized and summarized in Table 4.

TABLE 4
Cisplatin-encapsulated Low Molecular Weight Chitosan
Nanoparticles.

| | Diameter | | | |
|---|---|---|---|---|
| Sample Name | (nm) | PDI | KCPS | ZP (mV) |
| Blank | 265.3 | 0.538 | 165.5 | 12.1 |
| 0.75% cisplatin loaded NP | 150.6 | 0.305 | 254.5 | 16.8 |
| 1.87% cisplatin loaded NP | 187.8 | 0.212 | 316 | 14.4 |
| 3.75% cisplatin loaded NP | 306.6 | 0.474 | 276 | 13.7 |
| 7.5% cisplatin loaded NP | 315.9 | 0.402 | 377.9 | 14.5 |
| 15% cisplatin loaded NP | 285.4 | 0.324 | 314.8 | 14.7 |
| Blank + 2 mg Poloxamer | 235.8 | 0.324 | 294.8 | 15.2 |
| 188 | | | | |
| Blank + 4 mg Poloxamer | 255.5 | 0.57 | 392.2 | 14.2 |
| 188 | | | | |

Cisplatin-loaded nanoparticles containing different amounts of cisplatin were prepared using low molecular weight research chitosan are shown in Table 4. The PDI and the size controllability of using low molecular weight research grade chitosan did not perform well.

In order to obtain better quality drug-loaded nanoparticles, cisplatin-loaded nanoparticles were prepared using PROTASAN UP® CL 113 (chitosan chloride). These cisplatin-encapsulated nanoparticles prepared as described above were characterized and summarized in Table 5.

TABLE 5
Cisplatin-encapsulated PROTASAN ™ UP CL 113 (chitosan
chloride) Nanoparticles.

| | Diameter | | | |
|---|---|---|---|---|
| Sample Name | (nm) | PDI | KCPS | ZP (mV) |
| Blank | 133.5 | 0.221 | 313.9 | 44.38 |
| 0.75% cisplatin loaded | 122.3 | 0.284 | 278.5 | 40.35 |
| CHI-NP | | | | |
| 1.87% cisplatin loaded | 107.9 | 0.212 | 301 | 42.54 |
| CHI-NP | | | | |
| 3.75% cisplatin loaded | 74.2 | 0.276 | 332 | 37.33 |
| CHI-NP | | | | |
| 7.5% cisplatin loaded | 70.1 | 0.260 | 323.9 | 38.14 |
| CHI-NP | | | | |
| 15% cisplatin loaded CHI- | 75.1 | 0.279 | 354.1 | 36.92 |
| NP | | | | |
| 33.3% cisplatin | 143.8 | 0.237 | 364.4 | 38.79 |
| Loaded CHI-NP | | | | |

The best formulation was obtained with the 33.3% cisplatin-loaded chitosan nanoparticles, and this was selected for further in vitro and in vivo studies. Briefly, 5 mL of tripolyphosphates (TPP) solution at 0.1% w/v containing cisplatin (5 mg) was added to a 10 mL acetic acid solution (0.175% v/v) containing 0.1% w/v chitosan, while stirring vigorously. The mixture was stirred continuously under room temperature for 10 min, yielding 33.3% cisplatin-loaded CHI-NP with the size of 143 nm.

The amount of cisplatin in cisplatin-loaded nanoparticles shown in Table 5 quantified by ICP-AES (Inductively coupled plasma atomic emission spectroscopy), and the loading efficiency, is shown in Table 6. The loading efficiency, plotted against the encapsulation efficiency is shown in FIG. 2.

TABLE 6
Quantification of Cisplatin in Cisplatin-loaded Nanoparticles by
ICP-AES).

| Loading eff. of | [symbol] conc. | Unencaps. conc. | Of cisplatin | Encaps. |
|---|---|---|---|---|
| cisplatin (%) | (ug/ml) | (ug/ml) | (ug/ml) | Eff. (%) |
| 0.75% | 7.5 | 3.24 | 4.26 | 56.8 |
| 1.87% | 18.75 | 6.32 | 12.43 | 66.3 |
| 3.75% | 37.5 | 11.72 | 25.78 | 68.7 |
| 7.5% | 75 | 21.83 | 53.17 | 70.9 |
| 15% | 150 | 47.7 | 102.3 | 68.2 |
| 33.3% | 333 | 89.9 | 243.1 | 73 |

[symbol]

indicates data missing or illegible when filed

The stability of cisplatin-loaded chitosan nanoparticles prepared from PROTASAN™ UP CL 113 in TABLE 5 was determined by measuring the diameter, PDI and Zeta potential of cisplatin-loaded chitosan nanoparticles at day 0 and at day 21 as shown in Table 7.

TABLE 7
Stability of Cisplatin-Loaded Nanoparticles

| Sample | Diameter | | Zeta | Diameter | | Zeta |
|---|---|---|---|---|---|---|
| Name | (nm) | PDI | (mV) | (nm) | PDI | (mV) |
| Blank | 133.5 | 0.221 | 44.38 | 135.4 | 0.233 | 45.12 |
| 0.75% | 122.3 | 0.284 | 40.35 | 128.7 | 0.231 | 43.12 |
| cisplatin | | | | | | |
| loaded NP | | | | | | |
| 1.87% | 107.9 | 0.212 | 42.54 | 111.2 | 0.278 | 40.11 |
| cisplatin | | | | | | |
| loaded NP | | | | | | |
| 3.75% | 74.2 | 0.276 | 37.33 | 75.25 | 0.29 | 38.21 |
| cisplatin | | | | | | |
| loaded NP | | | | | | |
| 7.5% | 70.1 | 0.260 | 38.14 | 72 | 0.273 | 37.1 |
| cisplatin | | | | | | |
| loaded NP | | | | | | |
| 15% | 75.1 | 0.279 | 36.92 | 77.5.5 | 0.263 | 35.59 |
| cisplatin | | | | | | |
| loaded NP | | | | | | |
| 33.3% | 143.8 | 0.237 | 38.79 | 141.3 | 0.254 | 37.01 |
| cisplatin | | | | | | |
| Loaded NP | | | | | | |

The data shows that the nanoparticles were stable for up to three weeks.

### Example 3

### Effect of pH on Cisplatin-Encapsulated Chitosan Nanoparticles Properties

Materials and Methods

The effect of pH on the size and zeta potential of the above mentioned 33.3% cisplatin-loaded chitosan nanoparticles was studied using a Zetasizer (Nano ZS, Malvern Instruments, UK). The aqueous dispersion of nanoparticles (12 ml) was titrated with 0.1 M sodium hydroxide solution (NaOH) under constant stirring over a range of pH (3.7-8). The titrated dispersion was transferred to a measuring capillary cell for the Zetasizer measurements. The changes in the properties (both size and charge) of the nanoparticles were measured as a function of pH.

Materials and Methods

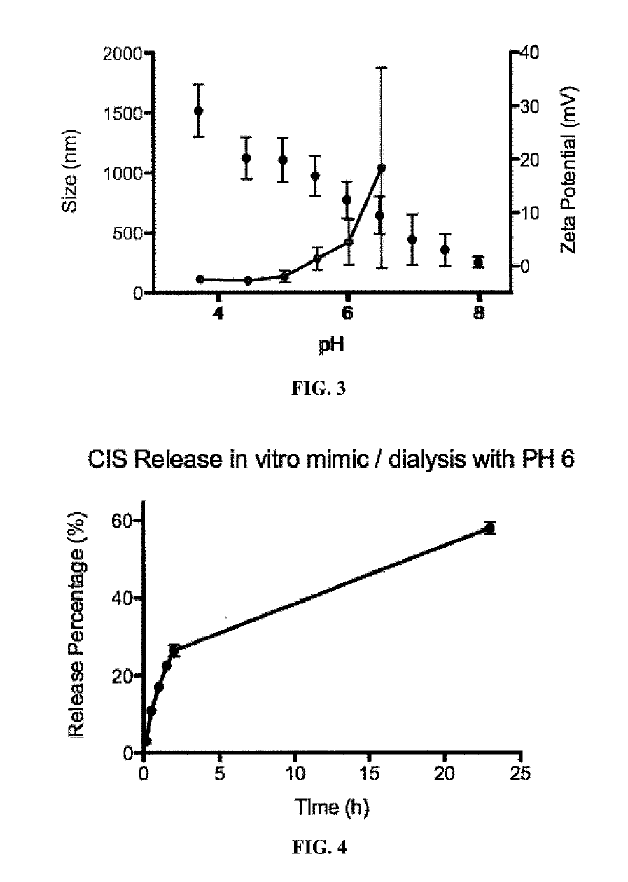

The change in zeta potential of nanoparticles over a pH range 3.7-8.0 is shown in FIG. 3 (in black). The high positive surface charge density for crosslinked chitosan at lower pH is due to the free surface amine groups of chitosan. As the pH of the nanoparticle suspension was increased, a greater proportion of amine groups were deprotonated, resulting in a decrease in the measured positive zeta potential for the particles.

The influence of pH on nanoparticle size is shown in FIG. 3. At a pH range of 3.7-5, the mean nanoparticle size was constant. The positive charge of chitosan in acidic medium results in repulsion between nanoparticles. However, as the pH was increased, the mean measured particle size increased, suggesting the nanoparticles had swelled and aggregated. The swelling of the nanoparticle results in the release of the drug at pH 6 or higher, which make the cisplatin-loaded chitosan nanoparticles a pH sensitive drug release system.

### Example 4

### In Vitro Mimicking Drug Release Study of Cisplatin-Encapsulated Chitosan Nanoparticles at pH=6

Materials and Methods

33% cisplatin-loaded chitosan nanoparticles were subjected to in vitro drug release studies. 3 mL of nanoparticles solutions were dialysed against pH 6 buffer. The rotating speed was fixed at 100 rpm. Samples of 5 ml each were withdrawn at specific time intervals (10 min, 30 min, 1 h, 1.5 h, 2 h and 24 h). The samples were collected and the cisplatin concentration was quantified by ICP-AES.

Results

The release percentage of cisplatin from nanoparticles was calculated and summarized in FIG. 4. Approximately 60% of the cisplatin was released from the nanoparticles after 24 h.

### Example 5

### Preparation of Chitosan Sponge Embedded with Cisplatin-Encapsulated Chitosan Nanoparticles

Materials and Methods

0.6 mL of 1% w/v citric acid containing 1% Chitosan w/v was added to 16 mL of either CHI-NP or cisplatin loaded CHI-NP (16:6 w/w NP and chitosan). The resulting solution was frozen at −80° C. and lyophilized for 2 days to obtain a nanoparticle embedded sponge. Citric acid was used both as a permeation enhancer as well as a taste-masking agent.

In order to understand the release profile of nanoparticles from the sponge-like matrix, FITC-labeled nanoparticles were synthesized. Briefly, 25 mg of Chitosan was dissolved in 25 ml of (0.175%, v/v) acetic acid aqueous solution and the pH value was adjusted to 6.0 with 1 M NaOH. One mg of fluorescein sodium salt was dissolved in 100 μl of ethanol and added into the chitosan solution. To catalyze the formation of amide bonds, EDAC [1-ethyl-3-(3-dimethylaminopropyl) carbodimide hydrochloride] was added to a final concentration of 0.05 M. The reaction mixture was incubated with permanent stirring for 12 h in the dark at room temperature. The resulting FITC conjugated chitosan was isolated by dialysis (cellulose dialysis tubing, pore size 12,400 Da; Seamless) against demineralized water. The evaluation of the derivatization process was performed by infrared spectroscopy (IR) and by spectrofluorimetry, using unmodified chitsoan and fluorescein as controls.

FITC-labeled nanoparticles were then obtained by following the same protocol using FITC-conjugated chitosan. FITC-labeled Nanoparticles were then embedded into the sponge as described above. The sponge was placed into 1 mL PBS and the solutions were collected at various time points. The release profile of nanoparticles from the chitosan sponge was measured over time by measuring the fluorescent intensity of the collected solution.

FITC labeled Cisplatin-encapsulated nanoparticles embedded sponge were prepared and placed into 6 well plate with two pH conditions, i.e., pH 5.5 and pH 7. The release of the nanoparticles from the sponge was measured by their fluorescent intensity at different time points using plate-reader.

Results

Nanoparticle release from the sponge increased with time as shown by FIG. 5. 90% of the nanoparticles were released from the sponge within approximately 20 min.

The results show the faster release profile at pH 5.5 than at pH 7, which indicates preferred release of our platform on tumor cells (more acidic than healthy cells).

### Example 6

### Cell Viability Studies Using Cisplatin-Loaded Chistosan Nanoparticles

Materials and Methods

Cell viability studies of 33% cisplatin-loaded nanoparticles were conducted using the MTT (3-(4,5-Dimethylthiazol-2-yl)-2,5-diphenyltetrazolium bromide) assay. FaDu (HTB-43, from the American Type Culture Collection, ATCC) cell line, which is a cisplatin-sensitive squamous carcinoma cell line derived from pharynx [Clin Cancer Res 10:8005-8017 (2004).], was utilized for in vitro study.

A cell viability study of 33% cisplatin-loaded nanoparticles was also conducted using the tetrazolium compound (3-(4,5-dimethylthiazol-2-yl)-5-(3-carboxymethoxy phenyl)-2-(4-sulfophenyl)-2H-tetrazolium inner salt, MTS. When incorporated by the cells, MTS is bioreduced by metabolically active cells. The amount of luminescence is directly proportional to the number of living cells in culture.

In a separate set of experiments, KB cells (a HeLa subline) were examined using the MTT assay following treatment with 15% cisplatin-loaded chitosan nanoparticles for 24 h.

To understand how the CIS-NPs effect a cisplatin-resistant cell line, cell viability studies of a cisplatin-sensitive ovarian cell line, A2670, and cisplatin-resistant ovarian cell line, A2670, were conducted.

Results

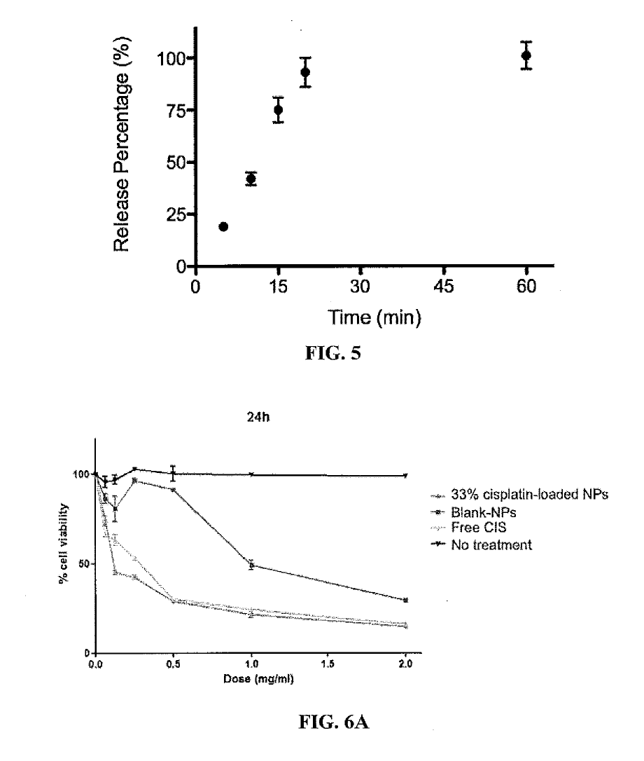

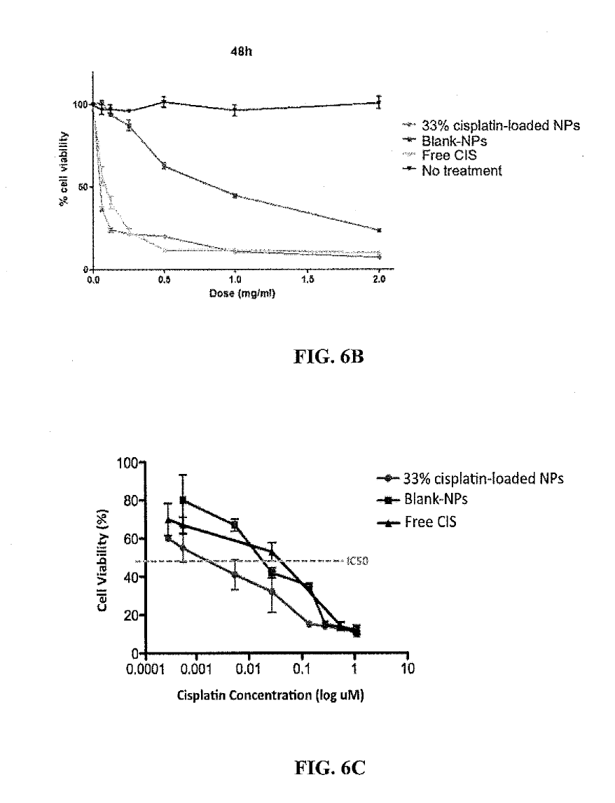

The results showing the in vitro therapeutic efficacy of free cisplatin, 33% cisplatin-loaded nanoparticles, and blank nanoparticles as assessed by the MTT assay against the FaDu cell line after 24 h and 48 h incubation are shown in FIGS. 6A and 6B, respectively.

Cell viability using the MTS assay of 33% cisplatin-loaded nanoparticles, blank nanoparticles or free cisplatin against HCPC 1 (hamster cheek pouch carcinoma) cells for 48 h is shown in FIG. 6C. The IC₅₀ values, the concentration at which 50% inhibition of cellular growth occurs, were around 0.06 μM for free cisplatin, 0.26 μM for blank nanoparticles and approximately 2.3 nM for 33% cisplatin-loaded nanoparticles.

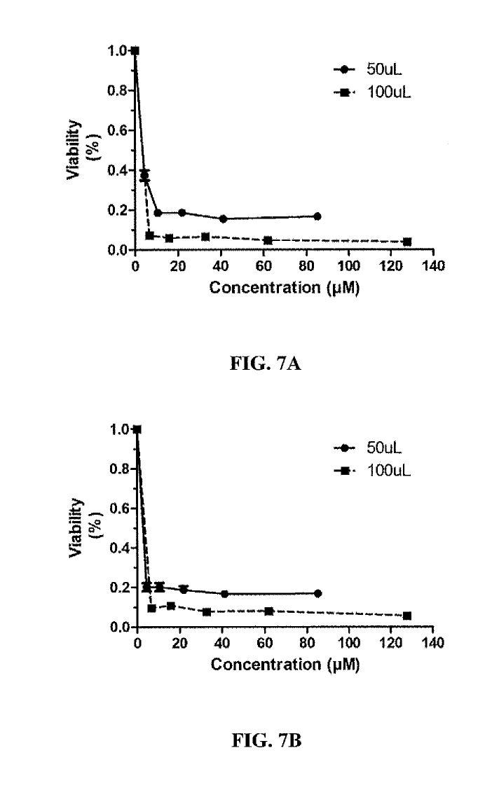

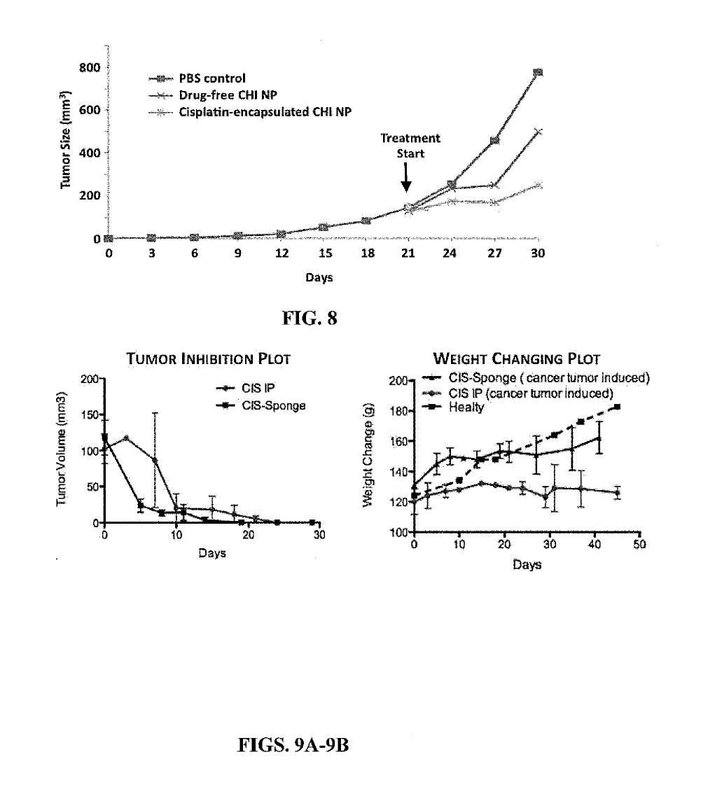

FIG. 7A and FIG. 7B show the results of a separate set of experiments, in which KB cells (a HeLa subline) were examined using the MTT assay following treatment with 15% cisplatin-loaded chitosan nanoparticles for 24 h (FIG. 7A) and 72 h (FIG. 7B). The results further validated the therapeutic efficacy of cisplatin-loaded chitosan nanoparticles. Besides KB cell line, which is a human oral epidermis, other types of cell lines (for example: 3T3: mouse fibroblast; A431: skin carcinoma) have been also investigated.

The studies with cisplatin sensitive and insensitive cells lines show a much lower IC₅₀ for CIS-NPs than free CIS for the sensitive cell line; however, there is an insignificant difference between the CIS-NPs and free CIS group on the resistant cell line. Although the cells can be destroyed in both groups on the resistant cell line by using high concentration of the drug, there is a dose-limiting toxicity issue for free CIS in in vivo case.

### Example 8

### In Vivo Mice Studies Using Cisplatin-Loaded Chistosan Nanoparticles

### A. FaDu Tumors

Materials and Methods

Nude mice, 4-5 week old, were anesthetized, shaved, and prepared for implantation of the tumor cells. FaDu cells were collected from culture, and 3×10⁵ cells suspended in a 1:1 mixture of PBS buffer and Matrigel were then injected subcutaneously into the back of a mouse. After 21 days when tumors reached approximately 150 mm³ in size, mice were divided into 3 groups of five mice, minimizing weight and tumor size difference. Tumor-bearing mice were treated by subcutaneous injection of PBS, cisplatin-loaded nanoparticles, or drug-free chitosan nanoparticles (1.15 mg/kg cisplatin equivalent). Two doses were administrated with 3-day interval, i.e. at day 21, day 24, day 27 and day 30, respectively. After injections, the animals were monitored closely, and measurements of the tumor size and body weight for each animal were performed at regular intervals using calipers without knowledge of which injection each animal had received. The tumor volume for each time point was calculated according to the formula, (length)×(width)²/2, where the long axis is the length, the short axis is the width.

Results

Median tumor growth curves prepared for each group depicted the median tumor size as a function of time (FIG. 7). The results demonstrate that the cisplatin loaded nanoparticles reduced tumor size.

The tumor tissues of the three groups (CIS NPs intratumoral, free cisplatin intratumoral and free cisplatin intravenous) were collected at the endpoint of the treatments (three weeks) and analyzed using ICP-MS. The results show the group treated with nanoparticles have the most drug accumulation in the tumor versus the other two group, i.e., free cisplatin intratumoral and intravenous). The toxicity study in the blood of the mice shows there is minimal amount of chemo-drug in the blood stream of the nanoparticles intratumoral group compared to free cisplatin groups (both intratumorally and intravenously) within 24 h, suggesting the minimized toxicity of the delivery system.

### B. HCPC1 Tumors

Materials and Methods

Golden Syrian hamsters, 71-80 g, were anesthetized and prepared for implantation of the tumor cells. Hamster cheek pouch carcinoma (HCPC 1) cells were collected from culture, and 1×10⁸ cells suspended in PBS buffer were then injected subcutaneously into the cheek pouch of a hamster. After 7 days when tumors reached around 100 mm³ in size, hamsters were divided into 4 groups, minimizing weight and tumor size difference.

Tumor-bearing hamsters were treated by intraperitoneal injection of free cisplatin and topical placement of nanoparticles-embedded sponge (1.15 mg/kg cisplatin equivalent). Two doses were administrated with 3-day interval. After treatments, the animals were monitored closely, and measurements of the tumor size and body weight for each animal were performed at regular intervals using calipers without knowledge of which injection each animal had received. The tumor volume for each time point was calculated according to the formula, (length)×(width)²/2, where the long axis is the length, the short axis is the width. Tumor inhibition and weight changing percentage of HCPC1-allografted hamsters treated with CIS-NPs embedded sponge topically and free cisplatin intraperitoneally.

Results

The results shown in FIGS. 9A and 9B demonstrate that comparable decrease in tumor size was obtained with both treatments, but that weight loss was significantly less with the nanoparticles, demonstrating the greater safety with the same efficacy.

### Example 9

### Synthesis of PEG Conjugated Chitosan and PEGylated Chitosan Nanoparticles

Materials and Methods

PEG conjugated chitosan was synthesized as described below. 5 mL of acetic acid solution (0.175% v/v) containing 0.1% w/v chitosan was prepared and the pH was adjusted to 6 using 1 M NaOH. Subsequently, 5 mg of NHS-PEG-COOH was added into the chitosan solution at room temperature under magnetic stirring for 3 h. The mixture was then adjusted to pH 7. The reaction was performed overnight under an argon atmosphere. The resulting solution was then lyophilized to yield PEG conjugated chitosan.

To assess the chemical structure of chitosan conjugated to PEG, Polarized Fourier Transformed Infrared (FTIR) spectra were obtained for the characterization. The peak at 1713 cm⁻¹ characteristic to ester —C═O in PEG-NHS, which is disappeared in the FT-IR spectra of PEG-Chitosan due to ester bond convert to amide bond into PEG-Chitosan. Additionally, the intensity of the peaks at ≈1466 cm-1, ≈1 657 cm-1, ≈3365 cm-1 was increased due to the amide bond form in the cross linkage chitosan to the PEG. The results show the successful conjugation of PEG to chitosan.

PEGylated Chitosan Nanoparticles were prepared from PEG conjugated chitosan as described below. Briefly, 5 mL of tripolyphosphates (TPP) solution was added to a 10 mL acetic acid solution (0.175% v/v) containing 0.1% w/v PEG-chitosan, while stirring vigorously. The mixture was stirred continuously under room temperature for 10 min, yielding PEGylated CHI-NPs.

### Example 10

### Optimization of NP at Large Scale

Automation of processes is an indispensible part of industrial production as it enables both faster and cheaper manufacturing and standardization of product properties by eliminating batch-to-batch variations and human errors. It is especially necessary in nanotechnology applications where the product properties must be kept in very limited tolerances.

Materials and Methods for Automated NP Synthesis:

All the reagents and chemicals used are excipient or pharmaceutical grade.

Solution A: Solution A: 0.1% Cisplatin in 0.1% Tripolyphosphate (TPP) solution

Solution B: 0.1% Chitosan (CL113) in 0.175% acetic acid solution Place 10 mL solution B in a glass beaker and stir at 600 rpm on magnetic stirrer. Transfer a total of 10 mL Solution A drop wise on the stirred solution B with the help of a peristaltic pump or any other pump that can provide a constant flow rate, as used herein at 1.5 mL/min, but which could be modified to yield a different size, charge, polydispersity, NP yield, and drug encapsulation efficiency properties.

Different ratios of solutions A to solution B (A:B) were used from 1:1 (as in the above case) to 1.1:0.85. Different mixing ratios produce different NP properties. Gradually increase the stirring speed of solution B to 650 rpm at the time when half of the solution A has been transferred. When transfer of solution A is completed, gradually increase the stirring speed to 700 rpm and then gradually add disaccharide trehalose to the solution to obtain a final trehalose concentration of 2%. Continue stirring until all the added trehalose has been dissolved (or for at least 10 min) to equilibrate the solution.

Measure the Z-average, the polydispersity index (PdI), the mean diameter of each peak, and NP yield (count rate) of the obtained NPs.

According to the required end product, either one of the below steps is followed.

For storage, the final NP solution is placed in a proper container and is frozen using liquid nitrogen, in dry ice, or in ultra low temperature freezer until complete freezing obtained and then they are freeze-dried until complete elimination of solvent obtained.

For placement in carrier wafers (ChemoThin wafer or CTW), then to the final NP solution, 2 mL 1% chitosan G113 solution in 1% acetic acid (or in 1% citric acid) is gradually added under stirring and the mixture is kept stirring for 10 min. Then this mixture is placed in a proper container and freeze-dried. If solution A to solution B ratio is different than 1:1, then the G113 chitosan amount to be added must be adjusted so that the volume of it is 10% of the total NP solution

Scalability is essential for mass production. Drug carrying nanoparticles are formed in solution environment by self-assembly, which is a dynamic process that takes place only under the correct chemical conditions. This technique is extremely sensitive to manufacturing variables including mixing rate of subsequent solutions and their concentrations, freshness, and purity. The mixing rate of subsequent solutions cannot be increased above a certain value without sacrificing nanoparticle properties. Furthermore, due to the dynamic nature of self assembly, the mixing process must be finished in a limited time as any delays in this duration increases the chance of deviation of nanoparticle properties or yield from optimal values. The sensitive nature of self-assembly production methodologies enforces adoption of strictly controlled batch type manufacturing processes and does not allow high production volumes per batch. By using the automation system that the team has developed, we have successfully adapted the original manual nanoparticle production processes into an automated version and it can now strictly control the properties of the resulting nanoparticle including size, polydispersity, and encapsulation efficiency. These parameters are of prime importance in terms of efficacy of the drug delivery system as well as raw material costs. Parameters can be adjusted to obtain a balance between maximal batch volume and nanoparticle yield (number of nanoparticles formed in a particular production batch) while keeping the properties of nanoparticles in narrow tolerances. Polydispersity of the obtained nanoparticles was found to be well within acceptable ranges. Initial results proved that a batch volume of up to 70 mL could be obtained without sacrificing nanoparticle properties or yield. Furthermore, it is possible to conduct these batch production processes in parallel to facilitate production speed.

Stability of the nanoparticle formulations is essential. Stability testing of the nanoparticle formulations showed they are stable in the production media up to 3 hours from production without decreasing the nanoparticle yield in the medium. Furthermore, it is possible to extend this at least up to 4 hours by adding natural disaccharide trehalose into the production media without any decrease in the yield.

The method involves a freeze-dry step. This could be a concern when it comes to the stability of NP after the process. In order to prove that NP remains the similar structure and properties after freeze-dry, powder form of NPs obtained after freeze-dry were re-suspended into different pHs. The size of the NP at different pH solutions was measured by Zetasizer after 30 min. The increased percentage of NP size was calculated as followed: (the size of NP in different pHs minus the size of NP before freeze-dry)/(size of NP before freeze-dry)*100.

Purification of the NP solution, i.e., remove all the excess compound, such as TPP and free cisplatin. At a small scale process, which uses 30 K PALL Nanosep Filtration Device has proved to be successful. More than 90% of the excess components were removed and results in highly purified NP product. As for the purification at a large scale, the team worked with PALL Life Science on a Minimate TFF (tangential flow filtration) system. The result shows the excess of free compound is removed from 100 ml within 1 h.

### Example 11

### Loading Capacity and Cellular Uptake of Cisplatin Containing NPs

Materials and Methods

The formulation of NP has been optimized to achieve better drug loading capacity and encapsulation efficiency. The encapsulation efficiency (% EE) was determined by ICP-AES. Briefly, 400 uL of NPs were centrifuged using Pall Nanosep 30K filter (1100 ref, 8 min, 25° C.). After centrifugation, the bottom solution (corresponding to the free cisplatin) and the top solution (corresponding to the NPs) were collected and the amount of Pt in these solutions was measured by ICP-AES after 1/100 dilution in Nitric Acid 2%. The EE % was calculated using the following formula:

100−(Pt bottom(mg)×100/Pt total theoretical(mg))

TR-146 cells were treated with FITC-labeled NP for 30 min at 52 uM and the cellular uptake of the NPs was measured by flow cytometry.

Results

The results demonstrate a loading capacity to 30% with 81% encapsulation efficiency.

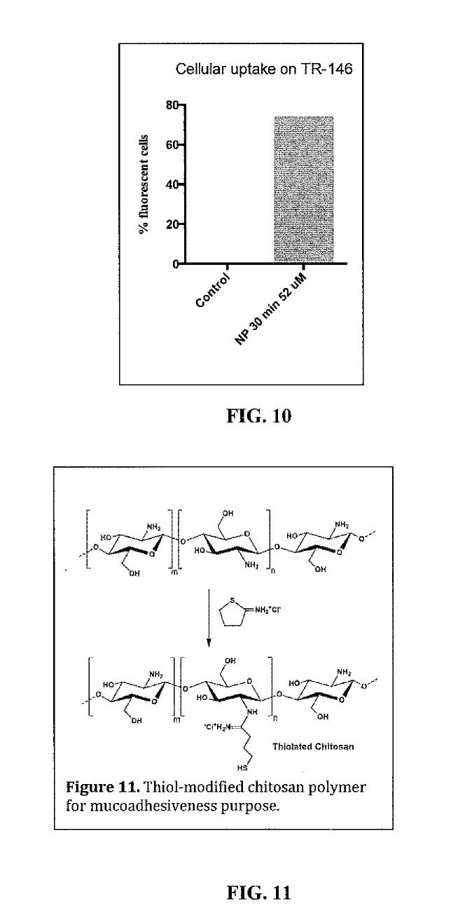

The results show around 75% of the NPs were taken up by the cells, as shown in FIG. 10.

### Example 12

### Modification of Chitosan

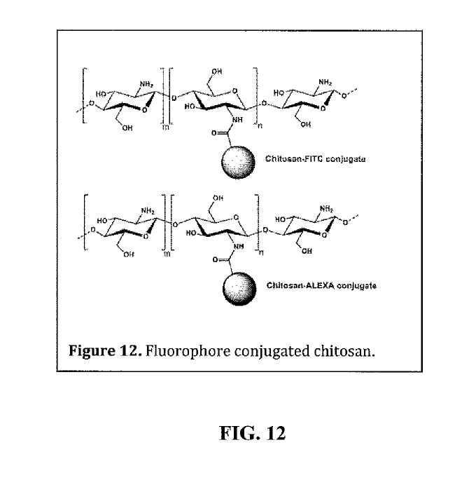

To increase the understanding of cellular uptake mechanisms of the chitosan nanoparticles and trafficking of these NPs in cytosolic compartments, chitosan polymers were synthesized with various fluorophores. Fluorophores (FITC, Alexa) were conjugated via the amine groups on the chitosan polymers, via ester chemistry (FIG. 11). Since amine groups of the chitosan polymer play a major role in nanoparticles formulations and drug encapsulation, only about 5% of amine groups were functionalized with the fluorophores. The chitosan polymer was also functionalized with thiol groups to improve mucoadhesive properties. 2-iminothiolane (FIG. 12) was used. Unlike chitosan-fluorophores, by using 2-iminothiolane, the positive charges were retained on the polymer, and thiol end groups were introduced.

Fluorescein sodium salt (FITC), 2-iminothiolane HCL, and N-(3-Dimethylaminopropyl)-M-ethylcarbodiimide hydrochloride (EDC) were purchased from Sigma-Aldrich. Alexa Fluor® 647 carboxylic Acid, succinimidyl ester (Alexa 647) were purchased from Life Technologies

Synthesis of Chitosan-FITC Conjugate:

25 mg of chitosan was dissolved in 25 ml of aq. acetic acid (0.175%, v/v) and pH of the solution adjusted to 6 with 1M NaOH. To this solution, 1 mg of fluorescein sodium salt in ethanol (10 mg/ml), and 21 mg of EDC were added and stirred at room temperature for 12 hours. After 12 hours, the reaction mixture was dialyzed against demineralized water for 2 days and freeze dried to achieve desired chitosan-FITC conjugate.

Synthesis of Chitosan-Alexa Conjugate:

25 mg of chitosan was dissolved in 25 ml of aq. acetic acid (0.175%, v/v) and pH of the solution of adjusted to 6 with 1M NaOH. To this solution, 50 ul of Alexa 647 in DMSO (1 mg/ml), and 21 mg of EDC were added and stirred at room temperature for 12 hours. After 12 hours, reaction mixture was dialyzed against demineralized water for 2 days and freeze dried to achieve desired chitosan-Alexa conjugate

Synthesis of Thiolated Chitosan:

50 mg chitosan was dissolved in 5 ml of 1% acetic acid and the pH of solution was adjusted to 6 with 1 M NaOH. To this solution 20 mg of 2-iminothiolane HCl was added and the reaction mixture was stirred at room temperature for 24 hours. After completion of the reaction, reaction mixture was dialyzed against 5 mM HCl, two times against 5 mM HCl containing 1% NaCl, 5 mM HCl, and finally against 0.4 mM HCl. After the dialysis reaction mixture was freeze dried to obtain solid thiol modified chitosan

### Example 13

### Expedited Synthesis of NPs Embedded Wafers in a High-Throughput Manner

Experiments have been performed using various formats for lyophilization, and the optimal one was found to be a 12-well plate.

Materials and Methods

### Reagents:

wafer solutions, liquid nitrogen, 12 well plates, lyophilizer.

### Protocol:

The cisplatin encapsulated NPs were first synthesized by ionic gelation method by the drop wise addition of cisplatin into the chitosan solution. The mixture was then stirred for 10 minutes. Following the stirring process, the chitosan glutamate (G113) solution in 1% acetic acid solution was then added into it and stirred for additional 1 minute. This mixture was then transferred into 12 well plates with 4 ml in each well. The freezing process was done in liquid nitrogen for 30 minutes, followed by lyophilization for 2 days.

Results

This platform maximizes reproducibility and allows precise control over both the shape of the wafer and the amount of drug in each wafer. Additionally, experiments have been conducted exploring alternate methods of freezing the wafer. It was found that freezing the wafer solution for 30 minutes in liquid nitrogen produced the same effects as freezing it for 3 hours on dry ice. The process yields 12 wafers a single one, and an 83% reduction in freezing time.

### Example 14

### Incorporation of Optimized Formulations of Sweeteners and Taste-Masking Agents to NPs Embedded Wafers

There is an unpleasant metallic taste associated with cisplatin. This unpleasant and often intolerable taste can dramatically decrease the quality of life of chemotherapy patients and poses a significant obstacle to topical administration of cisplatin. Rigorous experimentation was done to determine the optimal formulation of sweeteners and tastemasking agents that could be incorporated into the NP-embedded wafer protocol while minimizing modifications. Many flavor and sweetener formulations were tested under various conditions. The goal of this experiment was to determine the best combination of flavors/masking agents for concealing the metallic taste of cisplatin.

Materials and Methods

Six different flavor samples and one resolving agent were obtained from WILD Flavors. Powdered sucralose and EZ-Sweetz were purchased from Amazon.com, and Gentle Iron supplements were bought from Vitamin Shoppe.

Reagents and equipment used:

### Fruit Punch Flavor

### Tropical Punch Flavor

### Orange Cream Flavor

### Orange Creamsicle Flavor

### Peppermint Flavor

### Vanilla Mint Flavor

### Resolving Agent

25 mg Gentle Iron capsules (to test for cisplatin flavor)

### EZ-Sweetz

### Powdered Sucralose

Results

Each flavor was tested under 6 different parameters:

### 1 Drop Flavor+2 ml 5% Iron Solution

### 1 Drop Flavor+2 ml 5% Iron Solution+1 Drop EZ-Sweetz

1 Drop Flavor+2 ml 5% Iron Solution+1 Drop 42 mg/ml Sucralose Solution

### 1 Drop Flavor+2 ml 5% Iron Solution+1 Drop Resolving Agent

1 Drop Flavor+2 ml 5% Iron Solution+1 Drop EZ-Sweetz+1 Drop

### Resolving Agent

1 Drop Flavor+2 ml 5% Iron Solution+1 Drop 42 mg/ml Sucralose Solution+1 Drop Resolving Agent

Nearly all of the combinations resulted in a solution that completely covered up the strong metallic taste. When EZ-Sweetz was added to most of the flavors, it overpowered them to the point of only tasting sweetness. Some were so sweet; they were difficult to swallow. Additionally, the resolving agent had a fascinating effect on some of the flavors when combined with EZ-Sweetz. Not only did it tone down the sweetness, but 5 of the 6 combinations tested had their core flavors enhanced (for example, peppermint was much more pronounced) by the Resolver. The best results in 5 of the 6 flavors came from a combination of EZ-Sweetz and the Resolving Agent.

### Example 15

### Optimization and Incorporation of Permeation Enhancers into Wafer

This study was to determine the permeation depth up to which cisplatin encapsulated NPs embedded into a wafer can penetrate into the pig tongue. The platform technology can add various types of permeation enhancers on the surface of nanoparticles or mix into the sponge matrix. The flexibility of the platform can generate a library with various combinations of permeation enhancers that can permeate different depth of the tissue. Negative charged permeation enhancers, such as bile acid, can be added onto the surface of nanoparticles using layer-by-layer method.

Materials and Methods

FITC labeled chitosan NPs were used.

The following permeation enhancers (“PE”) were used:

### a) No PEs (Just PBS)

b) Ethanol (100% Ethanol diluted with 50% PBS)
c) 2-Dimethylaminopropionic acid dodecyl ester (DDAIP) (“DDAIP”) (2 g/100 ml diluted in PBS)
d) Ethanol+DDAIP (100% Ethanol diluted with 4 g/100 ml of DDAIP to achieve the same final working concentration of Ethanol and DDAIP as used in (b) and (c) respectively.
e) TDCA (100 mM diluted in PBS)

As an example, sodium deoxycholic acid (DCA) was chosen. 2.4 mL of TPP solution containing cisplatin (1 mg/mL) was slowly added into 3 mL of chitosan solution (acetic acid solution 0.175% v/v). The layer-by-layer coating of permeation enhancer (in this case, DCA) was done by quickly adding 60 uL of DCA (1.6 mg/mL) into the mixture. The solution was then stirred vigorously for 10 min at room temperature and the DCA-coated cisplatin-encapsulated nanoparticles (DCA-coated CIS-NPs) were obtained.

Alternatively, 1 mL of 1% w/v citric acid solution containing 1% chitosan and 10 mg of sodium deoxycholic acid (DCA) was added into 1 mL of CHI-NPs solution. The resulting solution was then frozen at −80° C. and lyophilized for 2 days in order to obtain a DCA/CIS-NPs-embedded sponge.

Fresh pig tongue was obtained from the Butcher shop at Cambridge. The middle portion of the tongue was selected for these studies. 5 designated sections were chosen on this portion and 0.75 mL of each of these PEs were applied at each of these sections, followed by the addition of the wafers (25 mg each) with additional 0.75 mL of PEs on top of the wafers. The incubation was done in the dark at RT for 1 hour. The tongue was then washed with water and all the wafers were removed from their designated sections. The respective sections were individually cut and embedded into an OCT for sectioning using a microtome. 50 microns slices were cut from each of these sections and 2 slices were placed on slides. The slides were kept submerged in water for 3 hours to remove any bound OCT as it interferes with the image quality. Following that, each of the slides from different sections were mounted with cover slips using a DAPI containing mounting media and imaged using a fluorescence microscope. Images were taken at 4×, 10× and 20× in both FITC and DAPI channels. In addition, slices of untreated tongue were also cut and imaged to account for inherent fluorescence of the tongue. However, before conducting permeation studies, many experiments were performed to discover the optimal concentrations of PEs that would not result in significant modifications to out current wafer synthesis protocol and allow seamless integration into the wafer.

Results

The results of the study showed that DDAIP is the most effective permeation enhancer (Table 8)

TABLE 8
Permeation Enhancers and Permeation Depth

| Permeation Enhancer - | Permeation Depth |
|---|---|
| (PE) (if any) | (microns) |
| Control | NA |
| PBS (No PE) | 70 |
| Ethanol | 140 |
| DDAIP | 200 |
| TDCA | 100 |
| Ethanol + DDAIP | 100 |

One of the main challenges in buccal pharmaceutical delivery is ensuring that the drug successfully bypasses the barriers of the mouth to reach its target site. The two most prominent barriers are the lipoidal barrier and the basement membrane. The lipoidal barrier is present in the upper epithelium and consists of membrane coating granules and tight junctions, while the basement membrane is much deeper in the epithelium and made up of extracellular matrix proteins that prevent large molecules from entering. While chitosan on its own is a permeation enhancer, it cannot penetrate deep enough into the epithelium (70 μm as shown in Table 8) for sufficient drug delivery. Its cationic properties interact with the negatively charged cell membranes, disrupting the lipoidal barrier and increasing permeation. When DDAIP-HCl was used as a PE, the maximum depth permeated by the NPs is 200 μm, well beyond the basement membrane barrier (around 100 μm). DDAIP-HCl is a hydrophilic molecule that has been shown to alter the dynamics of the cell's lipid-bilayer and loosen tight junctions between cells, increasing permeation. Studies have also shown that the DDAIP NPs follow a paracellular pathway of permeation, meaning that the NPs use the intracellular spaces between cells to travel deeper into the epithelium.

These results show that chitosan and DDAIP-HCl are able to overcome both the lipoidal barrier and basement membrane, penetrating the epithelium enough to reach even deep tumors.

Modifications and variations of the methods and materials described herein will be obvious to those skilled in the art and are intended to come within the scope of the appended claims. All references cited herein are specifically incorporated by reference.

We claim:

1. A formulation for delivery of an agent to a site in a mucosal cavity, the formulation comprising
a mucoadhesive polymeric matrix having nanoparticles containing therapeutic, prophylactic, diagnostic, or nutraceutical agent incorporated therein, which is taste masked or targeted to a tissue upon release in the mucosal cavity,
wherein the matrix has at least two surfaces, a first surface for contacting the site in the mouth for delivery of the agent, and a second surface exposed to the oral cavity,
wherein the first surface of the matrix comprises polymeric material having a molecular weight and density on the surface of the matrix to facilitate passage of the nanoparticles through the mucosa of the oral cavity, and
wherein the second surface of the matrix comprises a coating or film impermeable to passage of the agent to be delivered.

2. The formulation of claim 1 wherein the matrix comprises nanoparticles of chitosan or cyclodextrin having agent incorporated therein.

3. The formulation of claim 1 wherein the coating or film is formed of a polymer selected from the group consisting of hydropropylmethylcellulose, cellulose acetate, and polyacrylamides.

4. The formulation of claim 1 wherein the nanoparticles have a diameter between about 60 and 450 nm.

5. The formulation of claim 1 wherein the nano particles are coated with polyalkylene glycol moieties to enhance penetration through the mucosa to the site for delivery.

6. The formulation of claim 1 wherein the nanoparticles comprise targeting moieties on their surface to direct the nanoparticles to the site for delivery of agent.

7. The formulation of claim 1 wherein the matrix comprises attachment peptides effective to adhere the matrix to the tissue of the site where the agent is to be delivered.

8. The formulation of claim 1 wherein the agent is selected from the group consisting of chemotherapeutics, antiinfectives, anti-inflammatories, immunomodulators, vaccines, and combinations thereof.

9. The formulation of claim 8 wherein the antiinfectives are selected from the group consisting of antibiotics, antifungals, antivirals, and combinations thereof.

10. The formulation of claim 1 wherein the diagnostic agent is an imaging agent selected from the group consisting of iron oxide, gadolinium complex, radioisotopes, gold and combinations thereof.

11. The formulation of claim 1 wherein the agent is a platinum based chemotherapeutic.

12. The formulation of claim 1 further comprising one or more permeation enhancers.

13. The formulation of claim 12 wherein the permeation enhancer is selected from the group consisting of 2-Dimethylaminopropionic acid dodecyl ester, polyethyleneglycol, citric acid, bile salt, and beta-cyclodextrin.

14. The formulation of claim 1 comprising one or more taste-masking agents.

15. The formulation of claim 14 wherein the taste masking agent is selected from the group consisting of citric acid, sweeteners, and food flavoring agents.

16. The formulation of claim 1 further comprising an anti-inflammatory or antioxidant agent.

17. The formulation of claim 5 wherein the polyalkylene glycol moieties are polyethyleneglycol having a molecular weight of less than 10,000 Da.

18. The formulation of claim 6 wherein the moieties are RGD peptides.

19. The formulation of claim 1 wherein the polymeric matrix material is chitosan, the chitosan taste masks the agent to be delivered and the chitosan releases the nanoparticles upon exposure to the pH of the oral cavity.

20. The formulation of claim 19 wherein the nanoparticles release chemotherapeutic agents or diagnostic agents upon exposure to the pH of the oral cavity.

21. A method of delivering an agent to a site in a mucosal cavity comprising administering to the region the formulation of claim 1.

22. The method of claim 21 wherein the site comprises cancer cells.

23. The method of claim 21 wherein the site comprises oral, buccal or sublingual mucosa.

24. The method of claim 21 wherein the agent is delivered in a dosage not causing systemically effective levels of the agent.
